(19) United States
(12) Patent Application Publication
(10) Pub. No.: US 2006/0223071 A1
(43) Pub. Date: Oct. 5, 2006

(54) Methods, compositions, and kits for detecting nucleic acids in a single vessel

(76) Inventors: Michele Elizabeth Wisniewski, Johnson City, TN (US); William Mark Barbour, Kingsport, TN (US); Brendan William Boyd, Johnson City, TN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/096,775

(22) Filed: Apr. 1, 2005

(51) Int. Cl. C12Q 1/68; C12P 19/34

(52) U.S. Cl. 435/6; 435/91.2

(57) ABSTRACT

The invention encompasses processes, compositions, and kits for isolating and detecting nucleic acids from samples using a metal oxide coated onto a vessel. The nucleic acids can be processed and detected within this vessel.

### BACKGROUND OF THE INVENTION

1. Field of the Invention

The present invention relates to purifying, concentrating, and detecting nucleic acids in complex biological samples.

2. Related Art

In the field of nucleic acid detection, genetic analysis can involve the extraction and purification of nucleic acids from cells or tissues, amplification of the target nucleic acids, and detection of the sequence of interest.

There are many different ways to release genetic material from an organism. High temperatures or pressures can cause cells to lyse. Enzymatic approaches such as lysozyme and protease treatments aid removing critical membrane structures in cells and tissue to release cellular contents. Alkaline treatment and chaotropic salts can affect osmotic pressure outside the cells causing them to burst open.

Once the cells have been opened, the nucleic acid of the organism must be in a form suitable for detection. Some methods for preparing RNA or DNA are laborious using hazardous reagents to obtain purified substrates. For example, organic solvents such as phenol and chloroform can be used to lyse cells and to remove proteins and cell debris in the organic phase, leaving the nucleic acids in the aqueous phase. The RNA or DNA can be precipitated with an alcohol or spooled onto glass rods to concentrate the nucleic acid for downstream processing.

A crude purification of nucleic acids can also be done by a “salting out” technique that precipitates proteins with a high concentration of LiCl, leaving the nucleic acids in the solution. Alternatively, purifying nucleic acids from cell debris can be accomplished by a using a CsCl gradient to purify nucleic acids based on their size. This method requires a high concentration of the nucleic acid substrate and expensive equipment, such as an ultracentrifuge.

Another technique employed for purifying nucleic acids employs an electropositive, hydrophilic solid matrix resin such as Silicon, Boron, or Aluminum to specifically bind nucleic acids in high salt and/or specific pH conditions. The DNA or RNA is eluted from the resins by changing the salt concentrations and pH.

For example, Boom et al. (U.S. Pat. No. 5,234,809) disclose the use of a nucleic acid binding solid phase of silica particles capable of binding the nucleic acid in the presence of a chaotropic substance. Similarly, Woodard et al. (U.S. Pat. Nos. 5,405,951, 5,438,129; and 5,438,127) disclose the use of binding matrixes having a hydrophilic surface, such as nitrocellulose, celite diatoms, silica polymers, glass fibers, magnesium silicates, silicone nitrogen compounds (e.g., SIN₄), aluminum silicates, and silica dioxide. The purified nucleic acid can be eluted with a mild buffer solution.

Efficient elution of the substrates from the solid support has been investigated (U.S. Pat. Nos. 5,523,392; 5,525,319; and 5,503,816). To date, glass and silica have been employed in different formats such as slurries precipitated by centrifugation, glass beads in a chromatography format, or silica coated iron oxides for a magnetic precipitation of nucleic acids extracted from solution.

Another method for purifying nucleic acids employs anion exchange resins. As described in U.S. Pat. Nos. 5,990,301; 6,020,186; 6,277,648B1, 6,609,618B2, and 6,674,371B1, resins coated with anion exchangers such as DEAE and DMAE bind specifically to nucleic acids in lysed biological samples. The binding of nucleic acids to this resin is dependent on the pH/salt concentration under which proteins and cell debris do not bind. The DNA or RNA can be eluted with a change in the salt and pH conditions.

Anion exchange chemistries have also been used in different formats to bind nucleic acids in solution. A widely use format is a chromatographic based system in which a sample flows through a column containing an anion exchange resin. A major problem with this format is that it cannot handle large particulates and requires removal of the cell debris by centrifugation. Another permutation of this technology is to coat iron oxide with silica and an anion exchanger, such as DEA, to bind the nucleic acids in biological sample, and then to precipitate the complex with a magnet (U.S. Pat. Nos. 6,368,800B1; 6,673,631; 6,027,945; 6,218,531B1; and 5,587,286). Recovery of the eluted DNA from anion exchangers yields at most 60% of the nucleic acids bound to resins.

The need for detecting few target molecules in large volumes that contain high levels of extraneous background DNA from complex biological samples has required new approaches to extract, purify, and concentrate nucleic acids for detection. Newer strategies involve higher affinity solid matrices for binding specifically to DNA or RNA.

These nucleic acid binding matrices include metal oxides such as magnesium, calcium, titanium, manganese, cobalt, nickel, zinc, yttrium, lanthanum, aluminum, iron, zirconium, and hafnium (EP 0391608A2). Metal oxides have a wide range of affinity for binding and extracting nucleic acids. Iron oxides appear to bind DNA or RNA with a relatively high affinity and have the property of reversible binding of nucleic acids under phosphate buffer conditions (U.S. Patent Appln. No. 2002/0068821 A1). Hydrated zirconium oxide beads, hafnium oxide beads, and aluminum oxide beads have been shown to have the ability to bind nucleic acids, which can then be easily eluted (EP 0897978 A2).

Metal oxides, such as zirconium, iron, cobalt, and hafnium oxides, bound to DNA can allow hybridization of probes to the bound nucleic acids (EP 0391698 A2). Moreover, aluminum oxide bound to DNA can permit hybridization of primers and amplification of the target by PCR (U.S. Pat. No. 6,291,166). In this patent, the unlabeled amplification products were detected by gel electrophoresis after removal from the PCR tube.

The rapid detection of pathogens in biological samples has become an important issue in clinical and environmental diagnostics. In order to detect low copy numbers of pathogens, further advances in sample preparation and detection methods must occur. Extremely efficient lysis methods for releasing the nucleic acids from the target organism could improve the sensitivity and accuracy of early detection. Likewise, the ability to perform preparation and detection steps in the same vessel could improve sensitivity and accuracy.

Thus, there exists a need in the art for new methods of preparing and detecting nucleic acids. The present invention fulfills this need.

### BRIEF SUMMARY OF THE INVENTION

The invention encompasses a method for the detection of a nucleic acid comprising attaching a nucleic acid molecule to a metal oxide coated onto a vessel; amplifying, in the vessel, a fragment of the nucleic acid molecule to generate an amplified product; and detecting, in the vessel, the amplified product. The invention further encompasses a method for the detection of a microorganism comprising lysing a microorganism to release a nucleic acid molecule; contacting the nucleic acid molecule with a vessel coated with at least one metal oxide; allowing the nucleic acid molecule to attach to the metal oxide; amplifying, in the vessel, a fragment of the nucleic acid molecule attached to the metal oxide to generate an amplified product; and detecting, in the vessel, the amplified product.

In one embodiment, the microorganism can be lysed with a reagent that cleaves the peptidoglycan bonds on the outer membrane, a protease, a detergent, and an alkaline solution.

Reagents that cleave peptidoglycan bonds include lysozyme, lysostaphin, or mutanolysin.

Proteases include proteinase K, pronase E, achromopeptidase, proteinase R, proteinase T, subtilisin DY, an alkaline serine protease from Streptomyces griseus or Bacillus lichenformis, dispase, subtilisin Calsberg, subtilopeptidase A, and thermolysin.

Detergents include t-octylphenoxypolyethoxyethanol (Triton X-100), polyoxyethylenesorbitan monolaurate (Tween-20), polyoxyethylenesorbitan monopalmitate (Tween-40), polyoxyethylenesorbitan monostearate (Tween-60), polyoxyethylenesorbitan monooleate (Tween-80), polyoxyethylenesorbitan monotrioleate (Tween-85), (octylphenoxy)polyethoxyethanol (IGEPAL CA-630), triethyleneglycol monolauryl ether (Brij 30), and sorbitan monolaurate (Span 20).

Alkaline solutions include 50 mM-500 mM KOH or NaOH.

In another embodiment, the microorganism is lysed with a chaotropic salt or a cationic surfactant. Chaotropic salts include guanidinium salts, such as guanidine hydrochloride, guanidine thiocyanate, guanidinium chloride, guanidinium hydrochloride, and guanidinium isothiocyanate.

Cationic surfactants include cetyltrimethylammonium bromide (CTAB), cetyltrimethylammonium chloride (CTACl), tetradecetyltrimethylammonium bromide (TTAB), tetradecetyltrimethylammonium chloride (TTACl), dodecyltrimethylammonium bromide (DTAB), dodecyltrimethylammonium chloride (DTACl), dodecylethyldimethylammonium chloride (DEDTAB), decyltrimethylammonium bromide (D10TAB), and dodecyltripheylphosphonium bromide (DTPB).

Metal oxides include an oxide of aluminum, titanium, zirconium, hafnium, scandium, yttrium, lanthanum, vanadium, tantalum, chromium, molybdenum, tungsten, boron, gallium, indium, germanium, tin, zinc, nickel, magnesium, and iron.

Vessels include a tube, column, plate, and well. In a preferred embodiment, the vessel is a PCR tube.

In one embodiment, the attached nucleic acid molecule is washed with a buffer that comprises t-octylphenoxypolyethoxyethanol (Triton X-100), polyoxyethylenesorbitan monolaurate (Tween-20), polyoxyethylenesorbitan monopalmitate (Tween-40), polyoxyethylenesorbitan monostearate (Tween-60), polyoxyethylenesorbitan monooleate (Tween-80), polyoxyethylenesorbitan monotrioleate (Tween-85), (octylphenoxy)polyethoxyethanol (IGEPAL CA-630), triethyleneglycol monolauryl ether (Brij 30), sorbitan monolaurate (Span 20), methanol, ethanol, or isopropenol. In a preferred embodiment, the buffer comprises at least 5% w/w ethanol and at least 0.5% w/w polyoxyethylenesorbitan monolaurate (Tween-20).

In one embodiment, the amplified product is detected by hybridization to a probe. Probes include a labeled DNA, PNA, RNA, o-methyl RNA, LNA, or modified nucleotide probe. The probe can be fluorescently labeled.

In another embodiment, the amplified product is detected with an intercalating dye. In another embodiment, the nucleic acid molecule can be amplified with a fluorescently labeled primer.

In one embodiment, the nucleic acid molecule is amplified by the polymerase chain reaction (PCR). In one embodiment, the amplified product is detected during amplification. In another embodiment, the amplified product is detected after amplification.

The invention encompasses a kit for detecting nucleic acids in a biological sample comprising a vessel coated with at least one metal oxide; instructions for binding a nucleic acid molecule to the metal oxide; and instructions for amplifying and detecting, in the vessel, a nucleic acid molecule bound to the metal oxide.

In another embodiment, the invention encompasses a kit for detecting nucleic acids in biological sample comprising a vessel coated with at least one metal oxide and instructions for lysing a microorganism to release a nucleic acid molecule, contacting the nucleic acid molecule with a vessel coated with at least one metal oxide, allowing the nucleic acid molecule to attach to the metal oxide; amplifying, in the vessel, a fragment of the nucleic acid molecule attached to the metal oxide to generate an amplified product, and detecting, in the vessel, the amplified product.

The kit can include primers for amplifying a nucleic acid molecule bound to the metal oxide. The kit can also include a fluorescent probe for detecting a nucleic acid molecule bound to the metal oxide.

The kit can include a lysozyme, a protease, a nonionic detergent, and an alkaline solution.

### BRIEF DESCRIPTION OF THE DRAWINGS

The invention is more fully understood with reference to the drawings, in which:

FIG. 1A-C depict ethidium bromide stained agarose gels of PCR products amplified directly from Listeria genomic DNA bound to a metal oxide.

FIGS. 2A and B are graphs of real time PCR detection of a specific nucleic acid bound to PCR tubes coated with aluminum oxide pretreated with a basic solution verses no pretreatment prior to adding the genomic DNA.

FIG. 3 is a graph of real time PCR detection of a specific nucleic acid bound to PCR tubes coated with aluminum oxide using 800 mM guanidine to lyse open gram (+) micro-organisms.

FIG. 4 is a graph of real time PCR detection of a specific nucleic acid bound to PCR tubes coated with aluminum oxide using lysosyme, protease, a detergent, and an alkaline solution to lyse open gram(+) micro-organisms to increase the sensitivity of detection limits.

FIG. 5 is a graph of real time PCR detection of a specific nucleic acid bound to PCR tubes coated with aluminum oxide using lysosyme, detergent, and an alkaline solution to lyse open gram(+) micro-organisms to increase the sensitivity of detection limits.

FIG. 6 is a graph of real time PCR detection of a specific nucleic acid bound to PCR tubes coated with aluminum oxide using 800 mM guanidine to lyse open gram (−) micro-organisms.

FIG. 7 is a graph of real time PCR detection of a specific nucleic acid bound to PCR tubes coated with aluminum oxide using lysosyme, protease, a detergent, and an alkaline solution to lyse open gram(−) micro-organisms to increase the sensitivity of detection limits.

FIG. 8 is a graph showing the effect of lysozyme on lysis, capture, and detection of micro-organisms in a single vessel apparatus.

FIG. 9 is a graph showing the effect of protease on lysis, capture, and detection of micro-organisms in a single vessel apparatus.

FIG. 10 is a graph showing concentrations of primers and probes in the real-time PCR reaction for the detection of low copies of the specific nucleic acid.

FIG. 11 is a graph showing the effect of high concentration of primers and probes in the real-time PCR reaction to improve the detection of low copies of the specific nucleic acid.

FIG. 12 is a graph showing an improvement of detection of low copies of bacteria using Line-PCR which has the forward primer in higher concentration than the reverse primer.

FIG. 13 is a graph showing an improvement of detection of low copies of bacteria using Line-PCR which has the reverse primer in higher concentration than the forward primer.

### DETAILED DESCRIPTION OF THE INVENTION

The invention relates to novel methods, compositions, and kits for purifying and detecting nucleic acids. The invention encompasses preparing nucleic acids from complex biological samples, capturing the nucleic acids, removing inhibitors from the sample, and specifically amplifying and detecting the nucleic acids in a single vessel. The detection can be performed during (“real time detection”) or after (“end point detection”) the amplification reaction. The invention increases accuracy and sensitivity of detection of low copies of pathogens in large volumes while decreasing time and consumable cost to diagnostic laboratories.

### Methods

In one embodiment, the invention encompasses a process to release, extract, purify, and concentrate low copy numbers of nucleic acid substrates from a complex biological sample for the amplification and detection of a specific target.

In a preferred embodiment, the invention includes an efficient lysis method to extract the genetic material from the cells or tissues of interest. In one embodiment, the invention provides a novel protocol to lyse a biological sample and then capture, concentrate, and detect the genetic material on a solid matrix within a single vessel. The lysis protocol comprises a lysozyme treatment, a protease treatment, a detergent treatment, and a high ionic or alkaline condition to lyse open the cells.

In another embodiment, the invention includes a high affinity solid support to strongly and specifically bind the nucleic acids in the sample, but not to other cellular components. Preferably, metal oxides are used that have the property of high affinity binding to nucleic acids since they have a strong positive charge to attract the negatively charged phosphodiester backbone of RNA and DNA. The metal oxide binds the nucleic acids in the sample.

Elution of the substrate from the solid support is not required for direct detection of a specific target, since enzymatic and hybridization assays can occur on the target bound to the solid matrix. Furthermore, metal oxides have the advantage of binding RNA or DNA in a wide range of pH and salt concentration which is different from other solid supports consisting of silica or anion exchange resins. The nucleic acids bound to the metal oxide can be detected, in the same vessel, by an amplification of the genetic material in the presence of a labeled probe and a buffer that prevents further nucleic acids from binding to the solid matrix.

Nucleic Acids

Nucleic acids can be from any organism including animals and plants. Preferably, the nucleic acid is DNA, although RNA, especially rRNA or mRNA, can also be used.

Preferably, the nucleic acid is from a microorganism. Preferred microorganisms are bacteria, fungi, yeasts, and viruses.

Preferred bacteria include E. coli (e.g., E. coli O157:H7), Listeria (e.g., L. monocytogenes), Salmonella, Campylobacter, Legionella, (e.g., L. pneumophila) Bacillus (e.g., B. anthracis), Mycobacterium (e.g., M. tuberculosis), Mycoplasma, Clostridium, Chlamydia, Proteus, Enterococcus, Enterobacter, Pseudomonas, Neisseria, Staphylococcus (e.g., S. aureus), Streptococcus, Vibrio, Shigella, Helicobacter (e.g., H. pylori), Clostridium, Yersinia, Haemophilus, Pneumococcus, and Seratia. Preferred viruses include human immunodeficiency viruses (e.g., HIV-1), hepatitis viruses (e.g., Hepatitis B virus), herpesviruses, rhinoviruses, papillomaviruses, and adenoviruses.

Particularly preferred bacteria are pathogenic bacteria, especially those found in food products, such as E. coli O157:H7, Listeria, Salmonella, and Campylobacter.

Samples

Nucleic acids can be present in a variety of samples. Such sample types include biological samples such as blood, buccal swabs, mouth wash rinses, spinal fluid, hair follicles, mouse tails, nail follicles, tissue sections, urine, feces, and cervical fluids.

The samples can also be food samples, water samples, environmental sources, forensic sources, and amplification reactions. The samples can be swabs in a food processing plant. In a preferred embodiment, the sample is a food sample, such as a meat, poultry, or dairy product.

The samples can be manipulated prior to use in the invention, such as by filtering, purifying, disrupting tissues or cells, homogenizing tissues, centrifuging, precipitating, and/or adding reagents to the sample. The sample can be an amplified product.

Samples can also be cultures, such as tissue culture samples or fermented broths. For example, samples can be cultured in an appropriate growth medium for the microorganism of interest to increase the numbers of microorganisms for detection. In one embodiment, inhibitors of other microorganisms can be added to select for increases in a particular microorganism or set of microorganisms in the culture. For example, specific antibiotics or anti-mycotics can be added to the culture to prevent the growth of susceptible microorganisms. Various dyes (such as crystal violet and acriflavin), salts (such as sodium chloride and lithium chloride), and detergents (such as sodium dodecyl sulfate) can also be used as selective agents. Increased or decreased temperature and oxygen tension can also produce selective growth conditions.

Sample Lysis

Samples can be lysed to release the target nucleic acid of interest from the organism. Lysis can be performed prior to contact with a nucleic acid binding matrix. Alternatively, lysis can be performed in a vessel together with a nucleic acid binding matrix.

Many different lysis procedures can be used. Procedures can vary depending on the strength of the outer membranes of the organism of interest. In organisms that have highly resistant outer membranes, mechanical methods, such as a homogenizer, bead beater, French press, or sonicator can be used to burst open these samples. With most the biological samples such as tissue cultures, blood, and buccal cells, an alkaline or chaotropic salt treatment of the sample can be performed to lyse open the cells.

The chaotropic salt can be a guanidinium salt, for example, guanidine hydrochloride, guanidine thiocyanate, guanidinium chloride, guanidinium hydrochloride, or guanidinium isothiocyanate.

Other methods for lysis include; enzymatic procedures, boiling methods, organic solvent, and detergent treatments. Efficient lysis increases the detection of low copy number nucleic acids.

To achieve efficient lysis of pathogens and microorganisms, a very robust protocol has been discovered to lyse the cells, denature the genomic DNA, degrade the proteins, and emulsify lipids in a complex sample media. The method includes two enzymatic treatments: a reagent, such as a lysozyme, to cleave the peptidoglycan membranes, and a protease to degrade the proteins, especially nucleases, which can destroy the nucleic acids in the mixture.

In a preferred embodiment, the reagent used to cleave peptidoglycan bonds is selected from lysozyme, lysostaphin, and mutanolysin. In a particularly preferred embodiment, the reagent is 5 mgs/ml of lysozyme.

In a preferred embodiment, the protease is selected from proteinase K, pronase E, achromopeptidase, proteinase R, proteinase T, subtilisin DY, an alkaline serine protease from Streptomyces griseus or Bacillus lichenformis, dispase, subtilisin Calsberg, subtilopeptidase A, and thermolysin. In a particularly preferred embodiment, the protease is 1.5 mgs/ml of proteinase K.

In addition, two other treatments can be added to efficiently lyse a low level of pathogens: a detergent treatment to emulsify the lipids and denature the proteins and an alkaline treatment to burst open the cells in the sample and denature the genomic DNA.

In one embodiment, the detergent is a non-ionic detergent, for example, t-octylphenoxypolyethoxyethanol (Triton X-100), polyoxyethylenesorbitan monolaurate (Tween-20), polyoxyethylenesorbitan monopalmitate (Tween-40), polyoxyethylenesorbitan monostearate (Tween-60), polyoxyethylenesorbitan monooleate (Tween-80), polyoxyethylenesorbitan monotrioleate (Tween-85), (octylphenoxy)polyethoxyethanol (IGEPAL CA-630), triethyleneglycol monolauryl ether (Brij 30), or sorbitan monolaurate (Span 20). In a preferred embodiment, the nonionic detergent is polyoxyethylenesorbitan monolaurate (Tween-20) at 0.5%-2.0% v/v.

In another embodiment, the detergent is a cationic surfactant, for example, cetyltrimethylammonium bromide (CTAB), cetyltrimethylammonium chloride (CTACl), tetradecetyltrimethylammonium bromide (TTAB), tetradecetyltrimethylammonium chloride (TTACl), dodecyltrimethylammonium bromide (DTAB), dodecyltrimethylammonium chloride (DTACl), dodecylethyldimethylammonium chloride (DEDTAB), decyltrimethylammonium bromide (D10TAB), or dodecyltripheylphosphonium bromide (DTPB).

In a preferred embodiment, the alkaline solution is 50 mM-500 mM KOH or NaOH.

Performing lysis using a lysozyme/protease/detergent/alkaline solution protocol in the presence of a metal oxide solid support creates a highly sensitive and consistent nucleic acid sample preparation procedure that can be used for further molecular biology assays. Alternatively, the protocol can be performed without the protease treatment.

Metal Oxides

Nucleic acids can be bound to metal oxides for purification and detection, particularly by amplification of the nucleic acid bound to the metal oxide. The invention encompasses the use of metal oxides that have the property of being highly electropositive due to hydroxyl groups or other hydrophilic moieties. As used herein, the term “metal oxide” specifically excludes oxides of silicon.

Preferred oxides are oxides of aluminum, titanium, zirconium, hafnium, scandium, yttrium, lanthanum, vanadium, tantalum, chromium, molybdenum, tungsten, boron, gallium, indium, germanium, tin, zinc, nickel, magnesium, iron, and combinations of these metal oxides. Particularly preferred metal oxides in higher to lower order of affinity of binding/amplification/detection of the nucleic acid directly on the beads are aluminum, niobium, iron, zirconium, titanium, antimony, cerium, and germanium.

Metal oxides can be either magnetic or non-magnetic. Metal oxides are preferably 50% pure, more preferably 75%, 85%, 90%, 95%, or 99% pure.

In a preferred embodiment of the invention, the metal oxides are metal oxide beads. In one embodiment, the beads are between 1 and 200 microns in size. In another embodiment, the beads are between 2 and 150 microns in size, more preferably, between 5 and 100 microns in size. Most preferably, the beads are 5, 10, 25, 50, or 100 microns in size.

The metal oxide beads of the invention have the unexpected property of allowing the detection of an amplified product in the same vessel as a DNA is bound and amplified. This property allows for DNA purification, amplification, and detection in the same tube.

Solid Support

The metal oxide can be attached to a solid support, such as a plate, column, filter, beads, coating, or a rod. In a preferred embodiment, the metal oxide is attached to a vessel. As referred to herein, a vessel is a solid support with walls that can contain a solution in an amplification reaction. For example, the vessel can be a well or tube, most preferably a PCR tube. Preferably, the vessel is a plastic vessel. The vessel can include a top or lid.

In one embodiment, the support is coated with a layer of metal oxide, preferably aluminum oxide, at a 100-200 mesh. In another embodiment, the support is coated with metal oxide beads, preferably aluminum oxide beads.

To permit detection of amplification products in the same vessel, the concentration of beads must be sufficiently high to bind a detectable quantity of DNA and sufficiently low so as not to interfere with amplification and/or detection. Using higher concentrations of beads can lead to increased binding of primers or probes. Using higher concentrations of beads can also interfere with the ability to detect the signal in the same tube. The precise limits at which detection is no longer possible varies with the size and concentration of the beads in the tube, as well as the level of nucleic acid to be detected, and can be determined empirically, for example, by using the procedures set forth in the examples. As a standard for such determinations, the use of 15 mgs. of 100 micron aluminum hydroxide beads in a 250 μl PCR tube allows for detection of amplification products in the same vessel.

In a preferred embodiment, a vessel contains 5-50 mgs. of metal oxide beads in an area in contact with a 50 μl reaction volume. More preferably, the vessel contains 10-25 mgs. of metal oxide beads. Most preferably, the vessel contains 15 mgs. of metal oxide beads.

A vessel coated with a metal oxide includes a vessel having the metal oxide embedded in the vessel walls. Attachment can be performed using any method that attaches the metal oxide to the vessel and allows DNA binding to the metal oxide, while not interfering with amplification or detection. For example, attachment can be achieved using an adhesive, by chemical or vapor deposition, by propelling the metal oxide beads into a plastic vessel at high velocity, or by heating the metal oxide beads prior to contacting them with a plastic vessel. In one embodiment, metal oxide beads are heated to 800° C. prior to addition to a plastic vessel. In this manner, the beads are imbedded into the plastic vessel. Examples of methods for binding metal oxide beads to vessels can be found in WO/03020981, the specific methods of which are hereby incorporated by reference.

In a particularly preferred embodiment, the metal oxide is coated onto a vessel that allows for binding of nucleic acid to the metal oxide, amplification of the bound nucleic acid, and detection of the amplified products in the same vessel. Detection of the amplified products can be real-time (during the amplification process) or endpoint (after the amplification process). The invention allows the detection of amplification in the same vessel that is used for purification of nucleic acid. Detection of amplification in the presence of the metal oxide is a novel and unexpected result of the invention.

Nucleic Acid Binding to Metal Oxide Support

The sample conditions for binding nucleic acids to the metal oxide support encompass a wide range of aqueous conditions. This includes a broad range of buffered solution, broths, pH, and detergents. Preferred binding conditions are dictated by the lysis conditions that aid in promoting high affinity binding of nucleic acids to the metal oxide.

Preferably, binding of the nucleic acids is performed under alkaline conditions with a detergent. Preferentially, the binding/lysis buffer ranges in pH from 7-12, more preferably, 8-12. The binding buffer can contain a detergent, for example, non-ionic detergents, ionic detergents, and zwitterionic detergents, at a concentration of 0.1-25%. Preferably, 0.5-2% of Tween-20 is used in the binding/lysis buffer. After the binding/lysis buffer is added to the sample, the nucleic acid can be agitated over the metal oxide support by rotating, rocking, shaking, vortexing, or pipetting up and down to allow sufficient contact of the substrate with the solid support. Once the sample has been mixed/agitated thoroughly over the support, the sample can be removed and discarded.

Washing of Bound Nucleic Acids

The solid support bound with the nucleic acid can be subjected to a wash buffer to remove residual inhibitors from the sample, without dissociating the bound nucleic acid. Solutions for washing the solid support include buffered detergents, alcohols, and chelexing agents. The type and concentration of detergents can be similar to those described above. Preferred alcohols are methanol, ethanol, and isopropanol at a 20-70% concentration.

In one embodiment, the wash buffer includes a detergent, such as Tween-20, in a Tris pH8.0 buffer. The wash can be performed by adding the wash solution to the metal oxide then mixing/agitating the solution by pipetting, vortexing, rotating, or rocking. The wash can be performed one, two, three, or more, times. In a preferred embodiment, two washes are performed.

In one embodiment, the wash is performed with a buffer that comprises t-octylphenoxypolyethoxyethanol (Triton X-100), polyoxyethylenesorbitan monolaurate (Tween-20), polyoxyethylenesorbitan monopalmitate (Tween-40), polyoxyethylenesorbitan monostearate (Tween-60), polyoxyethylenesorbitan monooleate (Tween-80), polyoxyethylenesorbitan monotrioleate (Tween-85), (octylphenoxy)polyethoxyethanol (IGEPAL CA-630), triethyleneglycol monolauryl ether (Brij 30), sorbitan monolaurate (Span 20), methanol, ethanol, or isopropanol. In a preferred embodiment, the buffer comprises at least 5% w/w ethanol and at least 0.5% w/w polyoxyethylenesorbitan monolaurate (Tween-20).

Elution of Nucleic Acids from Support

Elution of the nucleic acid from the support is not necessary for the detection of pathogens. Rather, molecular biological reactions, such as PCR, which amplify the bound nucleic acids, can be performed on the solid support directly. However, if desired, the nucleic acids can be eluted from the support with elution buffers. Elution buffers can consist of Tris/EDTA pH 8.0, deionized water, or a phosphate buffer.

For high concentration of target substrate, elution will not interfere with downstream reactions, but for low concentration such as 1-100 copies of target sequence, elution may not be able to extract enough material for downstream processing. In a particularly preferred embodiment, no elution of the nucleic acid is performed.

Detection of Nucleic Acids on the Support

Nucleic acids bound to a support can be detected directly or can be detected during or after an amplification reaction.

### Direct Detection

When high copy numbers of specific nucleic acids are present in the sample, high copy numbers of the nucleic acids can be bound to the support. A high copy number of a specific nucleic acid can allow its detection directly on the support without any amplification of the nucleic acid. The nucleic acid can be directly detected with standard hybridization-based technologies, for example, using a radiolabeled, fluorescently labeled, chemiluminescently labeled, or enzymatically labeled probe.

### Nucleic Acid Amplification

With low copy numbers of pathogens, amplification of the specific target sequence can be performed to increase the number of copies of nucleic acid for detection.

“Amplification,” as referred to herein, means the generation of copies of a nucleic acid. A copy may comprise DNA, RNA, or a modified nucleic acid. Copies generated by an amplification method are referred to herein as “amplified product.”

Nucleic acids bound to metal oxides can be amplified with many varied amplification methods and amplification reaction conditions. For example, the amplification method can be rolling circle amplification (RCA), self-sustained sequence replication (3SR), multiple displacement amplification MDA, Nucleic acid sequence based amplification (NASBA), Transcription-mediated amplification (TMA), strand displacement amplification (SDA), Ligase chain reaction (LCR), b-DNA amplification, polymerase chain reaction (PCR, all forms including RT-PCR), ramification amplification RAM, loop-mediated isothermic amplification LAMP, isothermal and chimeric primer-initiated amplification of nucleic acids (ICAN), Isothermal Single Primer Amplification (SPIA), QB-replicase mediated amplification, or Invader assay.

A preferred amplification method is the polymerase chain reaction (PCR) amplification. See, e.g., PCR Technology: Principles and Applications for DNA Amplification (Ed. H. A. Erlich, Freeman Press, NY, N.Y., 1992); PCR Protocols: A Guide to Methods and Applications (Eds. Iinis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675.

In a particularly preferred embodiment, the amplification reaction is a Taqman™ real-time PCR reaction containing 1.5 mM MgCl₂, 200 μM dNTPs, 200 nM each primer pair, 200 nM Taqman™ probe, and 2 units of AmpliGold™ Taq with reagents using a thermocyler profile of 95° C. for 10 mins, and fifty cycles of 95° C. 15 secs and 55° C. for 1 min.

Other preferred amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), and nucleic acid sequence based amplification (NASBA) (U.S. Pat. Nos. 5,130,238, 5,409,818, 5,554,517, and 6,063,603). Other amplification methods that may be used are described in U.S. Pat. Nos. 5,242,794, 5,494,810, 4,988,617 and 6,582,938. The above references regarding amplification of nucleic acids are specifically incorporated by reference with respect to the disclosure therein of the specific reaction conditions used for amplification in each of the amplification methods.

### Detection of Amplified Product

In one embodiment, amplified product is detected by detecting the generation of double-stranded nucleic acid, such as with a fluorescent intercalating dye, e.g., SYBR green. In this embodiment, the signal generated by the intercalating dye corresponds to the level of amplified product. Thus, detection of a signal can represent the attachment of a specific nucleic acid to the support. In a particularly preferred embodiment, detection is performed during amplification. In another preferred embodiment, detection is performed after amplification.

In another embodiment, the amplified product is detected with a specific detection chemistry such as fluorescence resonance energy transfer (FRET) probes, Taqman probes, Eclipse MGB probes, molecular beacons, scorpion probes, fluorescently labeled primers, lightup probes or a dye-based chemistry, DNA, PNA, LNA, RNA, or o-methyl RNA, including modified bases that bind to the amplified product to detect the sequence of interest.

Detection of the amplified products can be real-time (during the amplification process) or endpoint (after the amplification process). The invention allows for detection of the amplification products in the same vessel in which the nucleic acid is bound to the metal oxide.

An enhancing reagent can be used to enhance amplification reactions by preventing the binding of primers, oligomers, or other amplification reagents to the solid support. This enhancing reagent contains a viscous component and a phosphate at a concentration sufficient to block the remaining solid support from binding nucleic acid reagents in the amplification reactions. Preferably, the viscous component comprises glycerol at 1-20% v/v. Preferably, the phosphate concentration is 20-200 mM.

The amplified nucleic acid can also be used for other molecular biology assays, including but not limited to, sequencing, cloning, restriction enzyme digest, RFLP, and any other manipulations of the nucleic acids.

### Compositions

The invention includes compositions for purification and detection of nucleic acids. These compositions are useful in the above-described methods.

For example, the invention includes reagents for the preparation of nucleic acids from cells as described above. The invention also includes reagents for the purification of nucleic acids, as described above. Such reagents include vessels, such as PCR tubes and wells coated with metal oxides.

In one embodiment, the composition comprises a 250 μl PCR tube coated with aluminum oxide beads. In a preferred embodiment, the beads are between 5 and 100 microns in size. In a particularly preferred embodiment, the tubes are coated with 15 mgs. of 5 micron beads. In one embodiment, the beads are imbedded into the walls of the PCR tube by heating them to 800° C. and contacting them with the tube. The coated tubes of the invention have the capacity to be used for DNA purification, amplification, and detection of amplified products in the same tube.

### Kits

The invention encompasses kits for the preparation, amplification, and/or detection of nucleic acids. The kit can include sample preparation reagents, amplification reagents, and/or detection reagents. The kit can include a reagent for lysis of a sample, which can be either in a solution or lyophilized form. The lysis reagent can contain lysozyme, protease, a detergent, and/or a high alkaline solution. The lysis reagent may be comprised of multiple components that are added to the sample at the same time or consecutively.

In a preferred embodiment, the kit contains nucleic acid capture and concentration vessel, for example, a PCR tube or a well, that has an aluminum oxide coating on the interior surface. The kit can include an enhancing reagent. The kit can include a wash solution and reagents for an amplification reaction. For example, the kit can contain PCR primers and probes specific for microorganisms of interest. Microorganisms of interest include E. coli (e.g., E. coli O157:H7), Listeria (e.g., L. monocytogenes), Salmonella, Campylobacter, Legionella, (e.g., L. pneumophila) Bacillus (e.g., B. anthracis), Mycobacterium (e.g., M. tuberculosis), Mycoplasma, Clostridium, Chlamydia, Proteus, Enterococcus, Enterobacter, Pseudomonas, Neisseria, Staphylococcus (e.g., S. aureus), Streptococcus, Vibrio, Shigella, Helicobacter (e.g., H. pylori), Clostridium, Yersinia, Haemophilus, Pneumococcus, and Seratia. Other microorganisms of interest include viruses, for example, human immunodeficiency viruses (e.g., HIV-1), hepatitis viruses (e.g., Hepatitis B virus), herpesviruses, rhinoviruses, papillomaviruses, and adenoviruses. In a preferred embodiment, the microorganism is a human pathogen.

The kit can include all, or some, reagents to lyse, purify, concentrate, amplify, and detect a specific nucleic acid in a complex biological sample. In a preferred embodiment, the specific nucleic acid is at a concentration of between 1 and 1000 copies/ml, preferably between 10 and 100 copies/ml. In one embodiment, the concentration of the specific nucleic acid is at least 100 copies/ml.

In a preferred embodiment, the kit will be able to purify, amplify, and detect nucleic acids in a single vessel. The kit can contain instructions for purifying, amplifying, and detecting nucleic acids in a single vessel.

The invention is more fully understood with reference to the following examples.

### EXAMPLE 1

### DNA Binding to Metal Oxides

DNA binding to different metal oxides was measured by placing 15 mgs. of less than 100 micron metal oxide particles in the presence of 10 or 1 million copies of Listeria monocytogenes genomic DNA. The DNA was diluted in 100 μl of 0.5% Tween-20 solution to the specific concentration. This solution enhances binding of nucleic acids to the metal oxides. The DNA/metal oxide slurry was rotated at room temperature for 10 mins. The particles were spun down to pellet the metal oxides and 34 μls of the solution were added to a real-time PCR reaction to quantitate the concentration of DNA that remained in solution unbound to the particles. The sequence of the primers and probes used in this experiment are shown below:

(SEQ ID NO:1)

Sequence 1: TCGTGCGCTTCTAGGT.

(SEQ ID NO:2)

Sequence 2: TGCTTTAGTTGCGATGGA.

(SEQ ID NO:3)

Probe Sequence: TATGAGTCGCCTTAGCTACAATGTATCT.

(SEQ ID NO:4)

Target Sequence: AATTACTAGATCAAACTGCTACAGGTGCTGCTA
CTCAAGTAAGCATCCAAGCGTCTGATAAAGCTAATGACTTAATCAATATC
GATCTTTTCAACGCTAAAGGTCTTTCTGCTGGAACAATCACTTTAGGTAG
TGGTTCTACAGTTGCTGGTTATAGTGCATTATCCGTTGCTGATGCTGATT
CTTCTCAACAAGCAACTGAAGCTATTGATGAATTAATCAATAACATTTCT
AACGGTCGTGCGCTTCTAGGTGCTGGTATGAGTCGCCTTAGCTACAATGT
ATCTAACGTGAACAATCAATCCATCGCAACTAAAGCATCTGCTTCATCCA
TTGAAGATGCAGATATGGCTGCTGAAATGTCCGAAATGACTAAATACAAA
ATTCTTACACAAACTTCTATCAGCATGCTTTCTCAAGCAAACCAAACACC
GCAAATGTTAACTCAATTAATTAACAGCTAA.

The Taqman™ real-time PCR reaction contained 1.5 mM MgCl₂, 200 μM dNTPs, 200 nM each primer pair, 200 nM Taqman™ probe, and 1.5 units of AmpliGold™ Taq using a thermocyler profile of 95° C. for 10 mins, and fifty cycles of 95° C. 15 secs and 55° C. for 1 min.

The percent of DNA bound to the metal oxides was determined by subtracting the concentration unbound from the total DNA concentration and then dividing by the total DNA concentration. Table 2 compares the binding affinities of different metal oxides. Iron, Aluminum, Zirconium, Antimony, and Cerium oxides bound >95% of Listeria monocytogenes gDNA at ten and one million copies/100 μls.

TABLE 2

| Metal | Size of | | | Metal | Size of | | |
|---|---|---|---|---|---|---|---|
| Oxide | particles | DNA | % Capture | Oxide | particles | DNA | % Capture |
| Al | 100-200 | 1 mil | 93.5 | Nb | 5 micron | 10 m | 56.4325 |
| | | | 92.4 | | | | 59.3737 |
| | | 100K | 99.6 | | | 1 mil | 39.7113 |
| | | | 100.0 | | | | 31.2332 |
| Al | 5 micron | 10 mi | 100.0 | Sb | 5 micron | 10 m | 99.9999 |
| | | | 100.0 | | | | 99.9999 |
| | | 1 mil | 100.0 | | | 1 mil | 100 |
| | | | 100.0 | | | | 99.9998 |
| Zr | 5 micron | 10 mi | 100.0 | Ge | 5 micron | 10 m | 33.0193 |
| | | | 100.0 | | | | 5.07552 |
| | | 1 mil | 99.9 | | | 1 mil | 64.1473 |
| | | | 100.0 | | | | 62.2098 |
| Sn | 5 micron | 10 mi | 37.7 | Ce | 5 micron | 10 m | 99.9804 |
| | | | 59.4 | | | | 99.8715 |
| | | 1 mil | 73.7 | | | 1 mil | 99.9069 |
| | | | 75.8 | | | | 99.9485 |
| Ti(III) | 5 microns | 1 mil | 76.1 | Fe(III) | ˜5 micron | 1 | 100.0 |
| | | | 64.4 | | | | 100.0 |
| | | 100K | 78.5 | | | 100 | 100.0 |
| | | | 39.8 | | | | 100.0 |

### EXAMPLE 2

### Amplification of DNA Bound to Metal Oxides

Amplification of the DNA bound to the metal oxide was examined using an ethidium bromide stained agarose gel. The DNA was bound to different metal oxides Aluminum (Sigma-Aldrich), Titanium (EM), Iron (SK Magnetics), Zirconium, Niobium, Germanium, Antimony, and Cerium (Alfa Aesar) in the presence of 40 mM NaOH and 0.5% Tween-20 solution. The alkaline solution was used to denature the DNA before binding to the metal oxides.

The DNA was titrated from 1 million copies/100 μls to 10 copies/100 μls in the presence of 15 mgs of particles. The DNA/metal oxide slurry was rotated at room temperature for 10 mins. The particles were spun down to pellet the metal oxides and the supernatant was discarded. The metal oxides were washed twice with a 0.5% Tween-20 solution and pelleted to discard the wash solution.

A 50 μl PCR reaction was add to the metal oxides bound with different concentrations of the genomic DNA. The reaction contained 1.5 mM MgCl₂, 200 μM dNTPs, 200 nM each primer pair, and 1.5 units of AmpliGold™ Taq with reagents using a thermocyler profile of 95° C. for 10 mins, and fifty cycles of 95° C. 15 secs and 55° C. for 1 min.

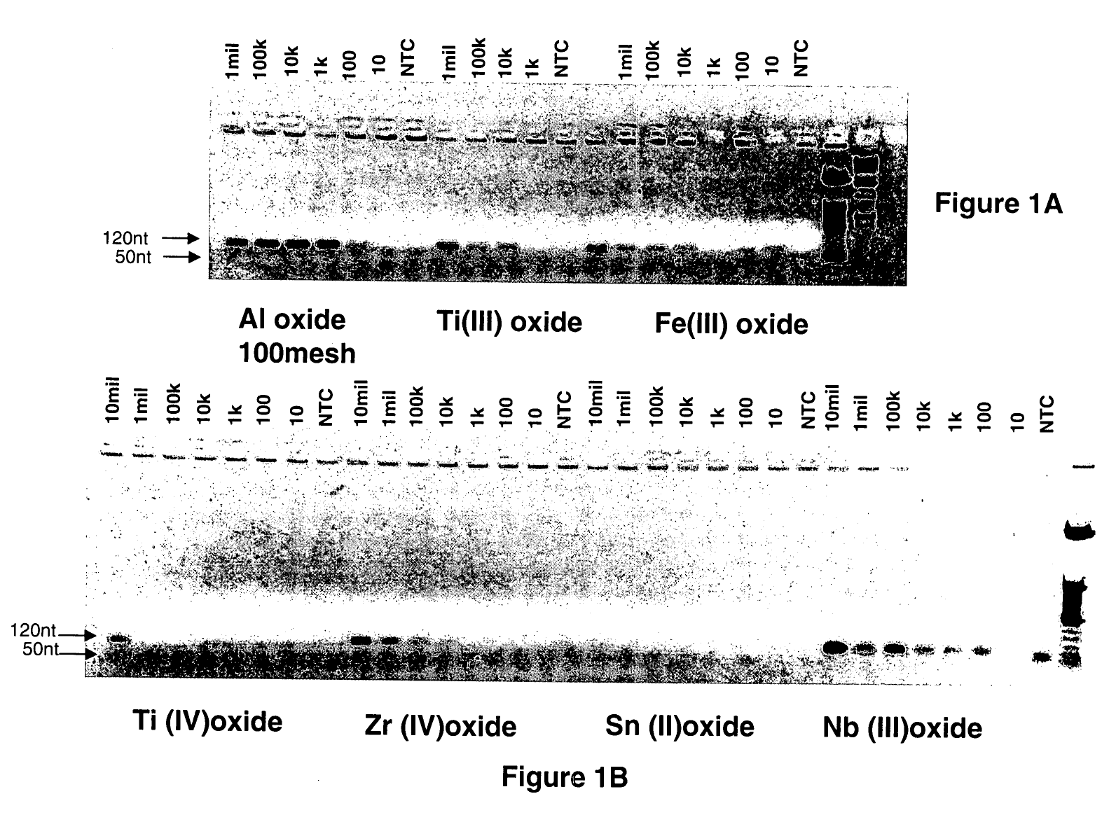

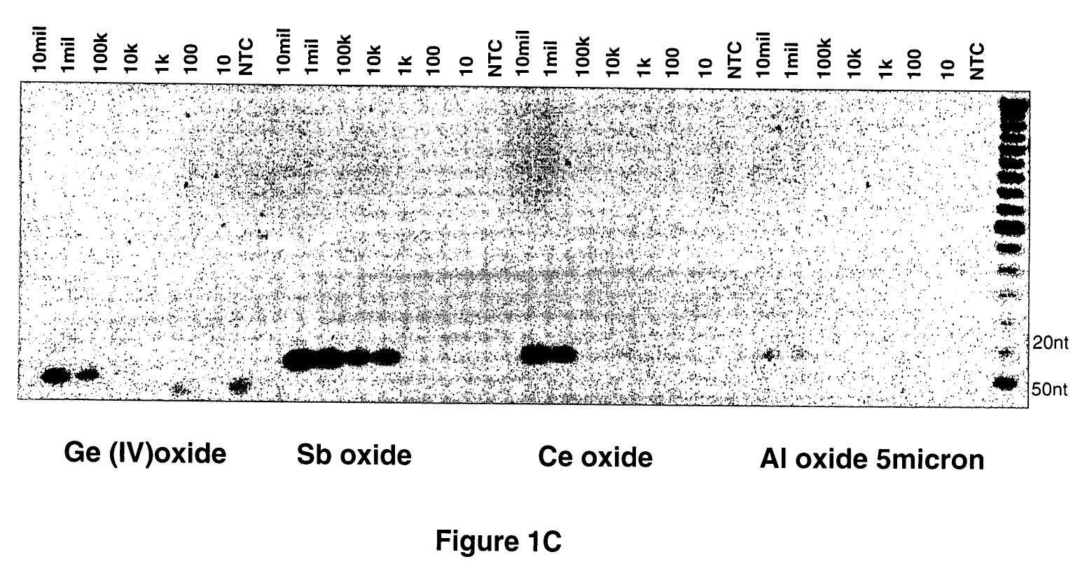

A glycerol/gel loading buffer was added to each PCR reaction and 18 μls of the PCR reaction was loaded onto a 1.2% agarose gel. An image of the agarose gel is seen in FIG. 1. Certain metal oxides allowed maximal amplification from the DNA bound to the solid support: Aluminum, Titanium, Iron, Zirconium, Niobium, Germanium, Antimony, and Cerium. Thus, amplification from the DNA bound to a solid support can occur efficiently.

### EXAMPLE 3

### Real Time Detection of DNA Binding to Aluminum Oxide Coated PCR Tubes

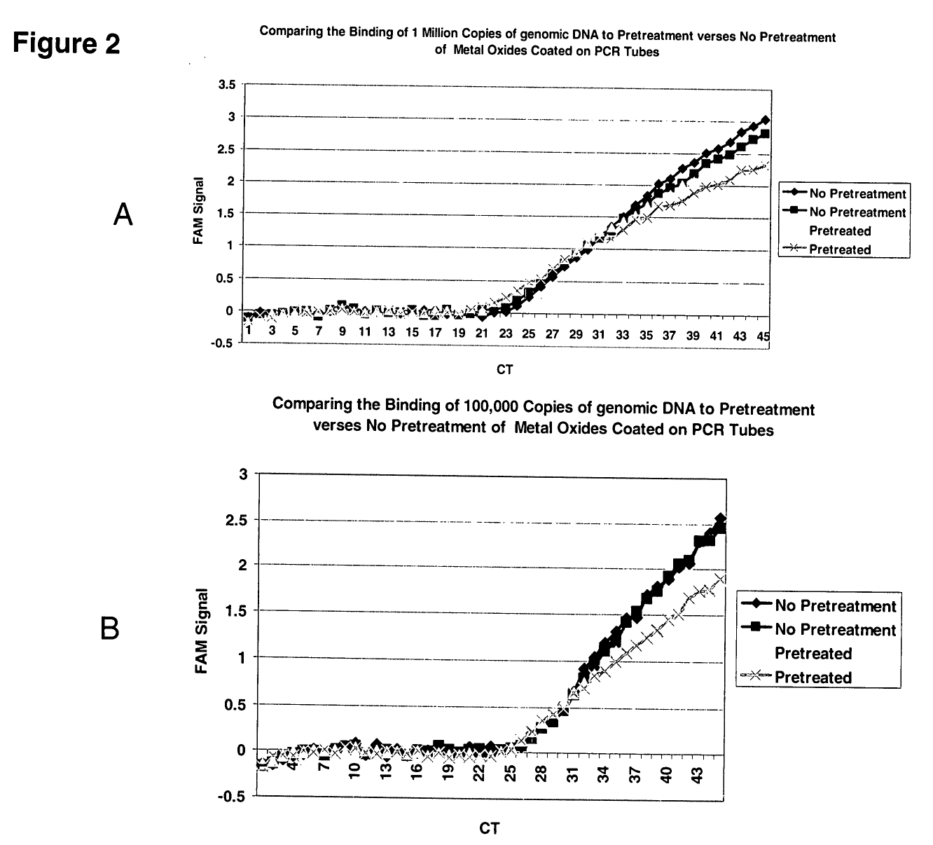

75-100 μls of a guanidine salt solution were incubated in a PCR tube coated with 15 mgs of 100-200 um aluminum oxide beads for 5 mins at room temperature to render the metal oxides hydrophilic. The genomic DNA was added after this treatment at different concentrations to determine the amount of DNA that is bound to the tubes. The sample was pipetted up and down 10 times and then discarded. The tube was washed and then PCR reagents were added on top of the solid matrix (FIG. 2).

Alternatively, genomic DNA was added straight to the tubes without rendering it hydrophilic. The sample was pipetted up and down 10 times and then discarded. The tube was washed two times with 200 μls of wash buffer (FIG. 2).

Each protocol was analyzed by a Taqman real-time PCR reaction containing 1.5 mM MgCl₂, 200 μM dNTPs, 200 nM each primer pair, 200 nM Taqman™ probe, Rox reference dye and 2 units of AmpliGold™ Taq with reagents using a thermocyler profile of 95° C. for 10 mins, and fifty cycles of 95° C. 15 secs and 55° C. for 1 min. Results are shown as a plot of cycle time vs. FAM signal.

The pretreatment of aluminum oxide is not necessary for DNA binding. The metal oxide does not need to be rendered hydrophilic before binding genomic DNA. Detection can occur in the same tube as the DNA binding and amplification reactions.

### EXAMPLE 4

### Improving Lysis of Bacteria Can Increase DNA Binding+ to Aluminum Oxide Coated PCR Tubes

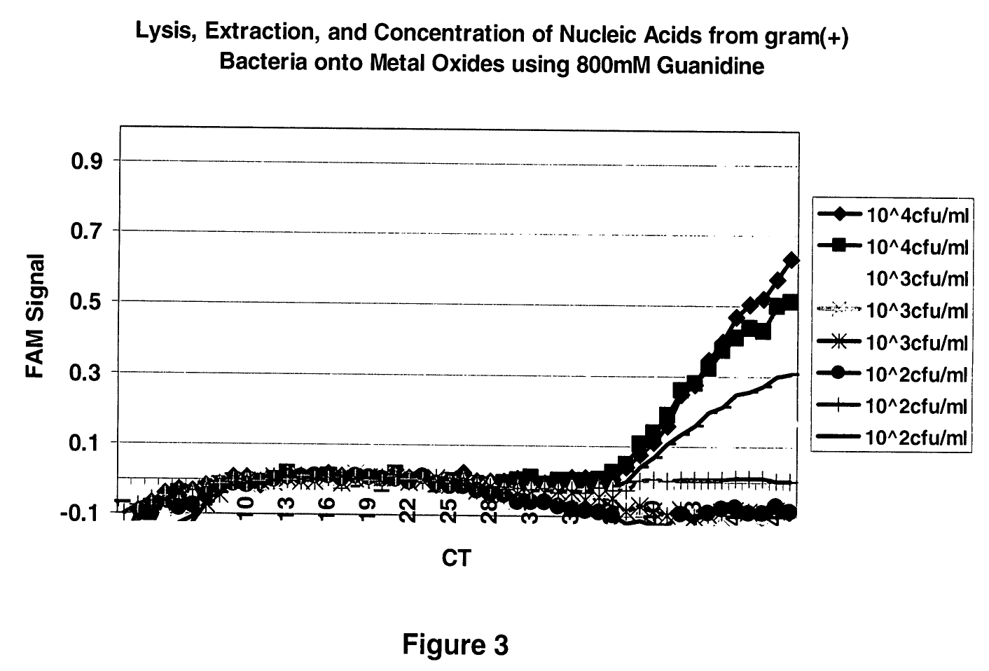

75-100 μls of a guanidine salt solution were incubated in a PCR tube coated with 15 mgs of 100-200 um aluminum oxide beads for 5 mins at room temperature. A food sample post-spiked with a predetermine concentration of Listeria monocytogenes was added to the tube, mixed by pipetting 5 times, and allowed to incubate at room temperature for 10 mins. The lysed sample was mixed 10 times over the aluminum oxide to capture the genomic DNA. The original sample was discarded and the aluminum oxide was washed two times with 200 μls of wash buffer (FIG. 3).

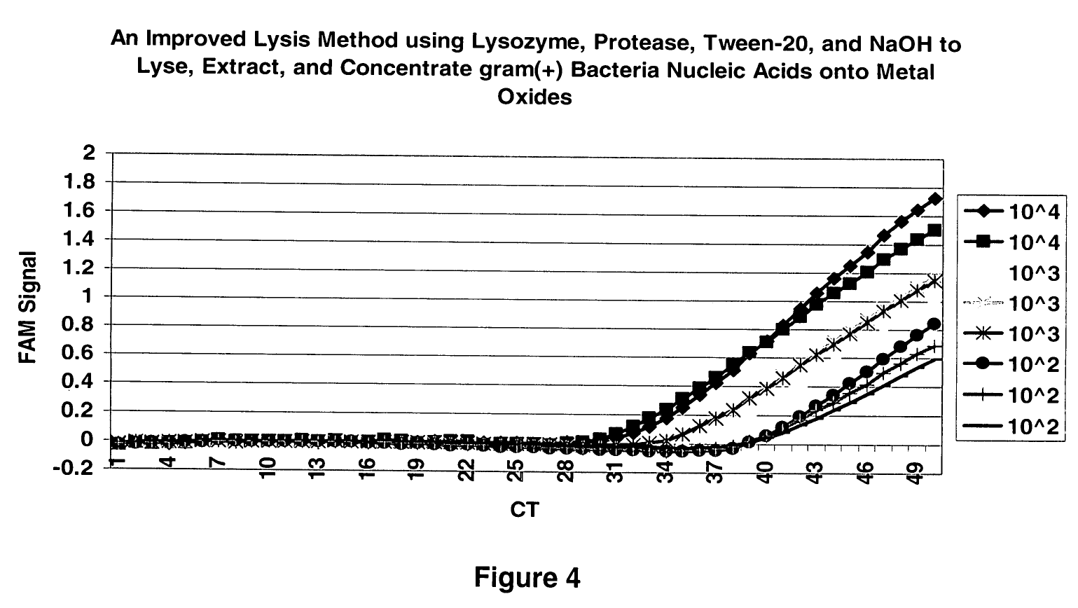

For comparison, 180 μls of a food sample post-spiked with a predetermined concentration of Listeria monocytogenes were added to the tubes and 5 mgs/ml of lysozyme. The sample was incubated 10 mins at room temperature. Next, 0.5% Tween-20 and 1.5 mgs/ml of proteinase K were added to the sample and was incubated for 20 mins at 55° C. 200 mM of NaOH was added and the sample was immediately pipetted 10 times, and then discarded. The tube was washed 2 times with a Tween-20 solution (FIG. 4).

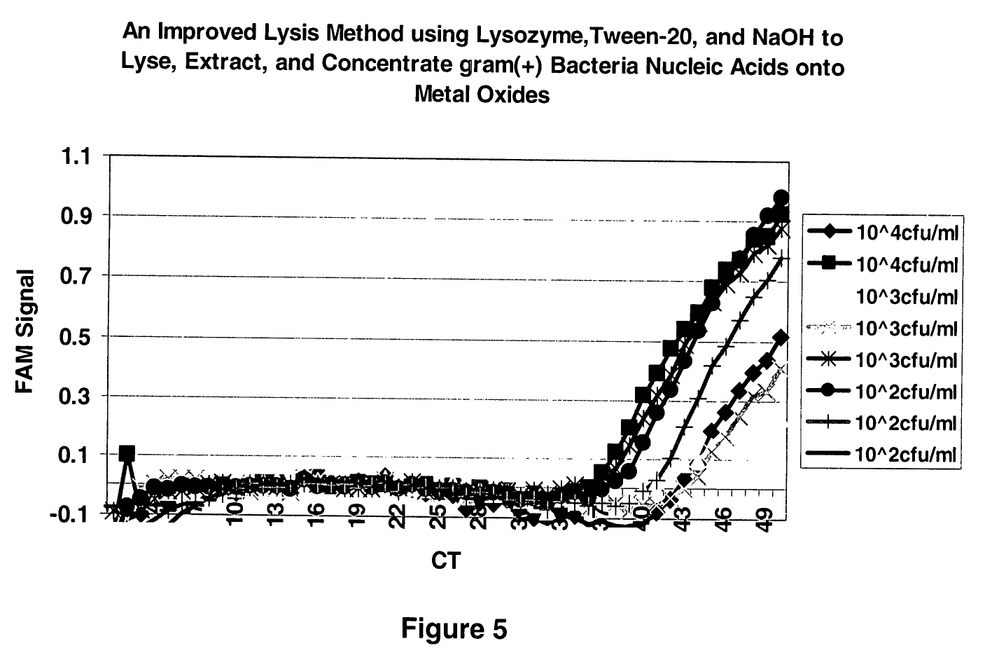

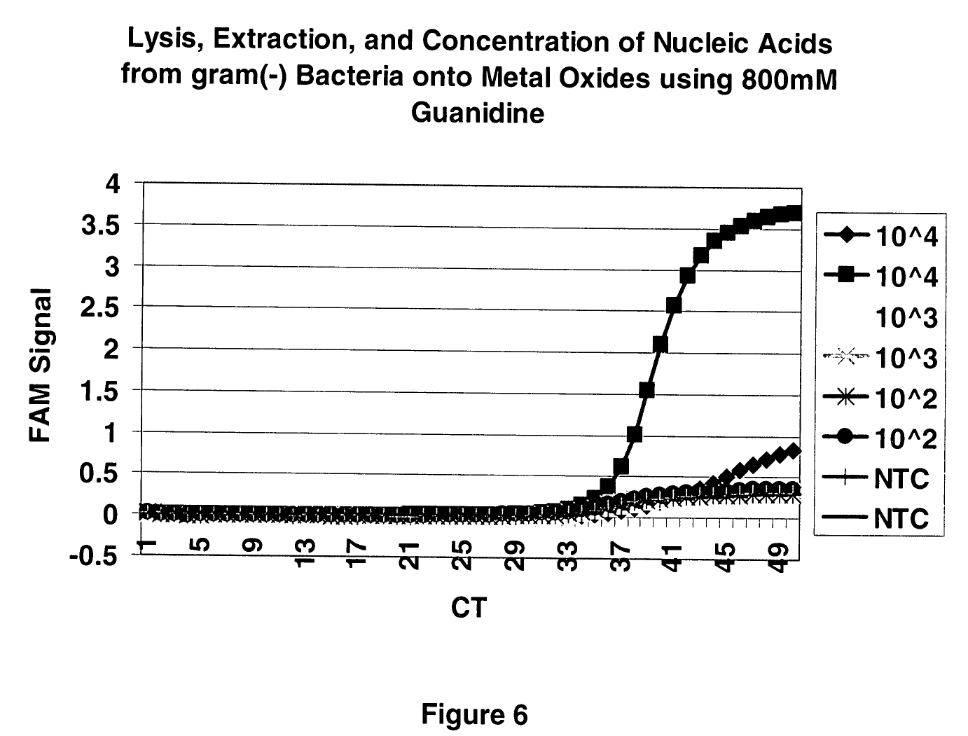

Alternatively, depending on the sample type and the amount of protein, 180 μls of sample were treated with only 5 mg/ml lysozyme for 10 mins at room temperature and no protease treatment was employed. The effect of the protease treatment was dependent on the fat content of the ground beef sample. 200 mM of NaOH and 0.5% Tween-20 was added to the sample and mixed by pipetting ten times in a PCR tube with aluminum oxide coating. The sample was discarded and the aluminum oxide bound with genomic DNA was washed 2 times with 120 μls of a Tween-20 wash solution (FIG. 5).

Each protocol was analyzed by a Taqman real-time PCR reaction containing 1.5 mM MgCl₂, 200 μM dNTPs, 200 nM each primer pair, 200 nM Taqman™ probe, and 2 units of AmpliGold™ Taq with reagents using a thermocyler profile of 95° C. for 10 mins, and fifty cycles of 95° C. 15 secs and 55° C. for 1 min. Results are shown as a plot of cycle time vs. FAM signal.

The lysozyme/protease/detergent/alkaline solution and lysozyme/detergent/alkaline solution protocols improved the ability to capture, concentrate and detect genomic DNA from gram(+) bacteria at low concentration.

### EXAMPLE 5

### Real Time Detection of E. coli O157:H7 DNA Binding to Aluminum Oxide Coated PCR Tubes

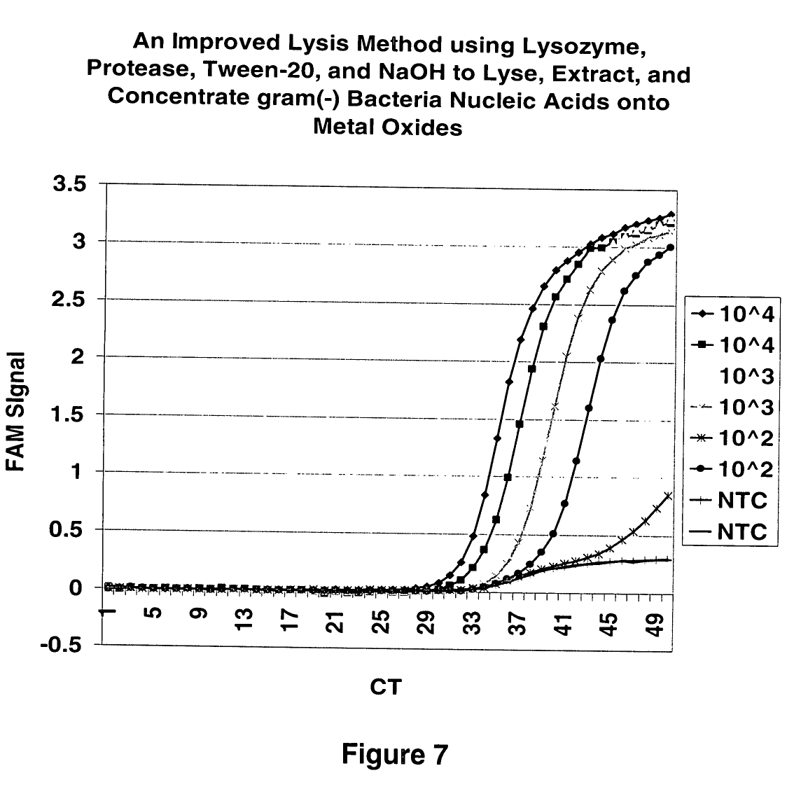

The same protocols were performed as in Example 3 using E. coli O157:H7. FIG. 7 is the improved lysis protocol with lysozyme/protease/detergent/alkaline solution. Each protocol was analyzed by a Taqman™ real-time PCR reaction containing 1.5 mM MgCl₂, 200 μM dNTPs, 200 nM each primer pair, 200 nM Taqman™ probe, and 2 units of AmpliGold™ Taq with reagents using a thermocyler profile of 95° C. for 10 mins, and fifty cycles of 95° C. 15 secs and 55° C. for 1 min. Results are shown as a plot of cycle time vs. FAM signal. The lysis protocol with lysozyme/protease/detergent/alkaline solution improved the ability to capture, concentrate and detect genomic DNA from gram(−) bacteria at low concentration.

### EXAMPLE 6

### Lysozyme Treatment Can Improve DNA Binding to Aluminum Oxide Coated PCR Tubes

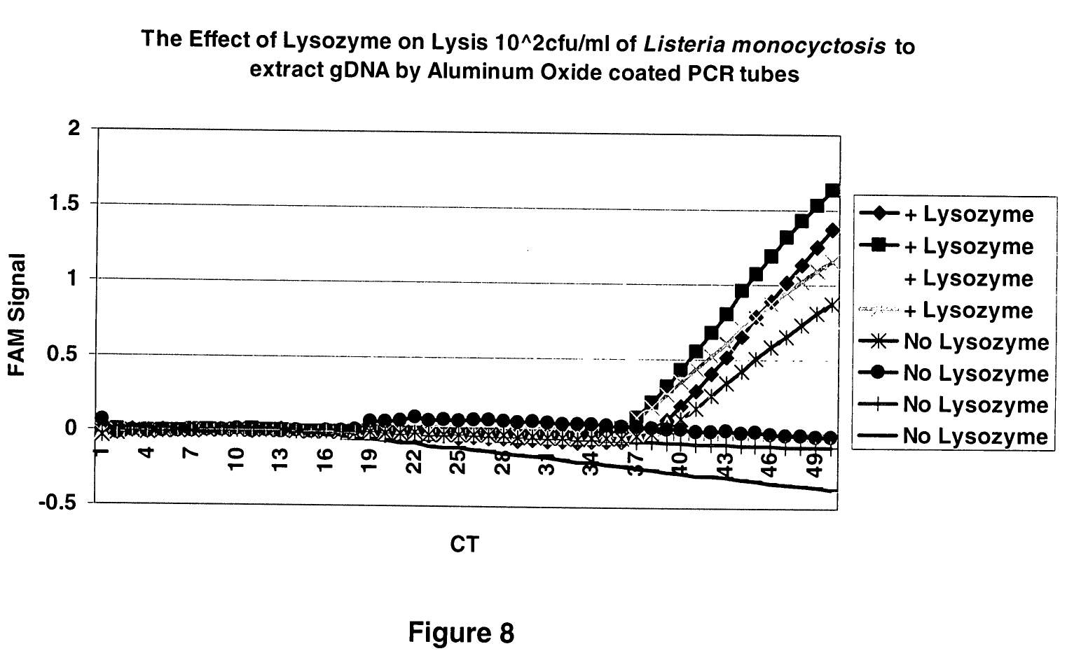

Lysozyme treatment can improve DNA binding to metal oxides. A protocol with and without lysozyme was performed to determine the effect of this enzyme. A 180 μl food sample, post-spiked with a predetermined concentration of Listeria monocytogenes, and 5 mgs/ml of lysozyme were added to tubes coated with 15 mgs of 100-200 um aluminum oxide beads. The sample was incubated 10 mins at room temperature. Next, 0.5% Tween-20 and 1.5 mgs/ml of proteinase K were added to the sample and was incubated for 20 mins at 55° C. 200 mM of NaOH was added and the sample was immediately pipetted 10 times, and then discarded. The tube was washed 2 times with a Tween-20 solution (FIG. 8).

Alternatively, 180 μls of sample were added to PCR tubes coated with aluminum oxide and treated with protease and 0.5% Tween-20 solution for 20 mins at 55° C. 200 mM of NaOH was added to the sample and mix by pipetting ten times in a PCR tube with aluminum oxide coating. The sample was discarded and the aluminum oxide bound with genomic DNA was washed 2 times with 120 μls of a Tween-20 wash solution (FIG. 8).

Each protocol was analyzed by a Taqman™ real-time PCR reaction containing 1.5 mM MgCl₂, 200 μM dNTPs, 200 nM each primer pair, 200 nM Taqman™ probe, and 2 units of AmpliGold™ Taq with reagents using a thermocyler profile of 95° C. for 10 mins, and fifty cycles of 95° C. 15 secs and 55° C. for 1 min. Results are shown as a plot of cycle time vs. FAM signal.

The ability to consistently extract, capture, and detect genomic DNA from bacteria was improved with lysozyme.

### EXAMPLE 7

### Proteinase K Treatment Can Improve DNA Binding to Aluminum Oxide Coated PCR Tubes

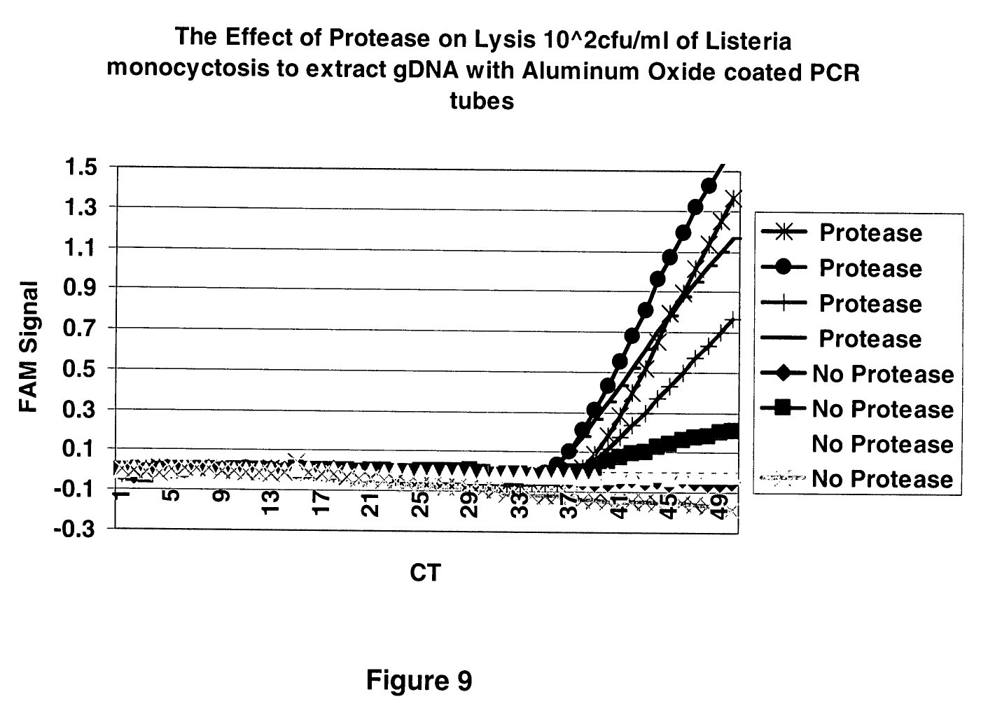

Protease treatment can improve DNA binding to metal oxides. A protocol with and without protease was performed to determine the effect of this enzyme. A 180 μl food sample, post-spiked with a predetermined concentration of Listeria monocytogenes, and 5 mgs/ml of lysozyme were added to the tubes coated with 15 mgs of 100-200 um aluminum oxide beads. The sample was incubated 10 mins at room temperature. Next, 0.5% Tween-20 and 1.5 mgs/ml of proteinase K were added to the sample and the sample was incubated for 20 mins at 55° C. 200 mM of NaOH was added and the sample was immediately pipetted 10 times, and then. The tube was washed 2 times with a Tween-20 solution (FIG. 9).

Alternatively, 180 μls of sample were added to PCR tubes coated with aluminum oxide and treated with 5 mgs/ml of lysozyme for 10 mins at room temperature. A final concentration of 200 mM of NaOH and 0.5% Tween-20 was added to the sample and mix by pipetting ten times in a PCR tube with aluminum oxide coating. The sample was discarded and the aluminum oxide bound with genomic DNA was washed 2 times with 120 μls of a Tween-20 wash solution (FIG. 9).

Each protocol was analyzed by a Taqman™ real-time PCR reaction containing 1.5 mM MgCl₂, 200 μM dNTPs, 200 nM each primer pair, 200 nM Taqman™ probe, and 2 units of AmpliGold™ Taq with reagents using a thermocyler profile of 95° C. for 10 mins, and fifty cycles of 95° C. 15 secs and 55° C. for 1 min. Results are shown as a plot of cycle time vs. FAM signal.

### EXAMPLE 8

### Primer Concentrations Can Improve Detection of DNA Bound to Aluminum Oxide Coated PCR Tubes

PCR primer and probes can be added at a high concentration in order to increase the ability to consistently lyse and detect micro-organisms in a single vessel. Micro-organisms were lysed by adding 180 μls of a food sample post-spiked with a predetermine concentration of Listeria monocytogenes and 5 mgs/ml of lysozyme to the tubes coated with aluminum oxide. The sample was incubated 10 mins at room temperature. Next, 0.5% Tween-20 and 1.5 mgs/ml of proteinase K were added to the sample and is incubated for 20 mins at 55° C. 200 mM of NaOH were added and the sample was immediately pipetted 10 times, and then discarded. The tubes were washed 2 times with a Tween-20 solution.

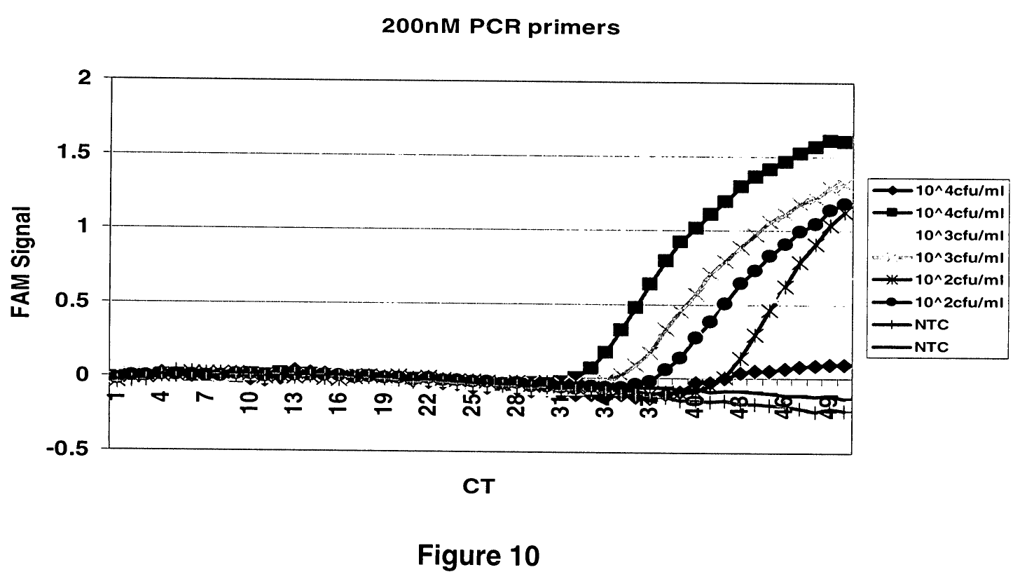

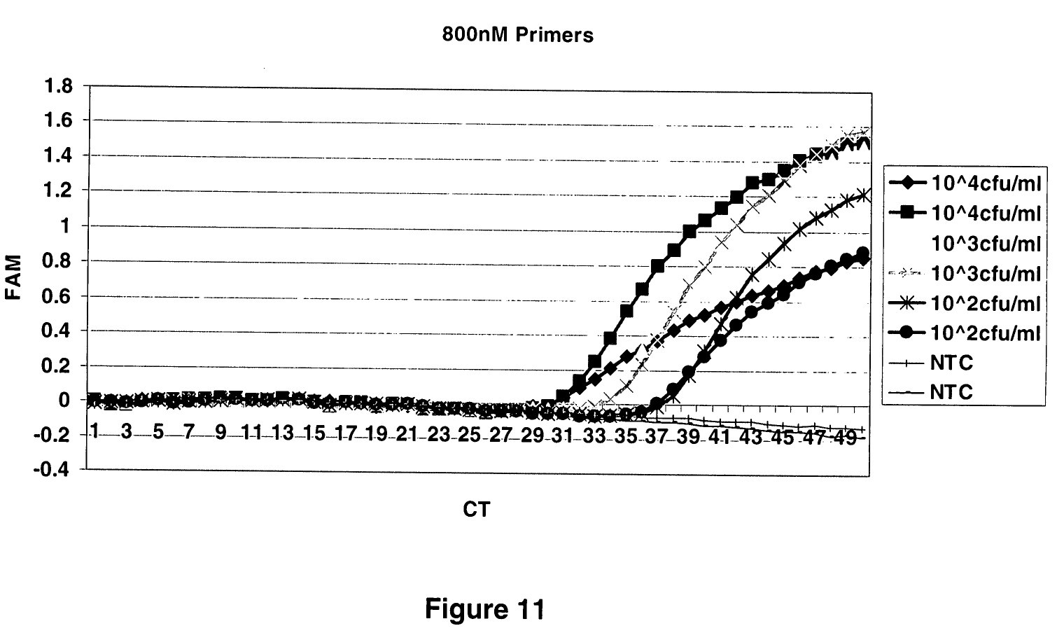

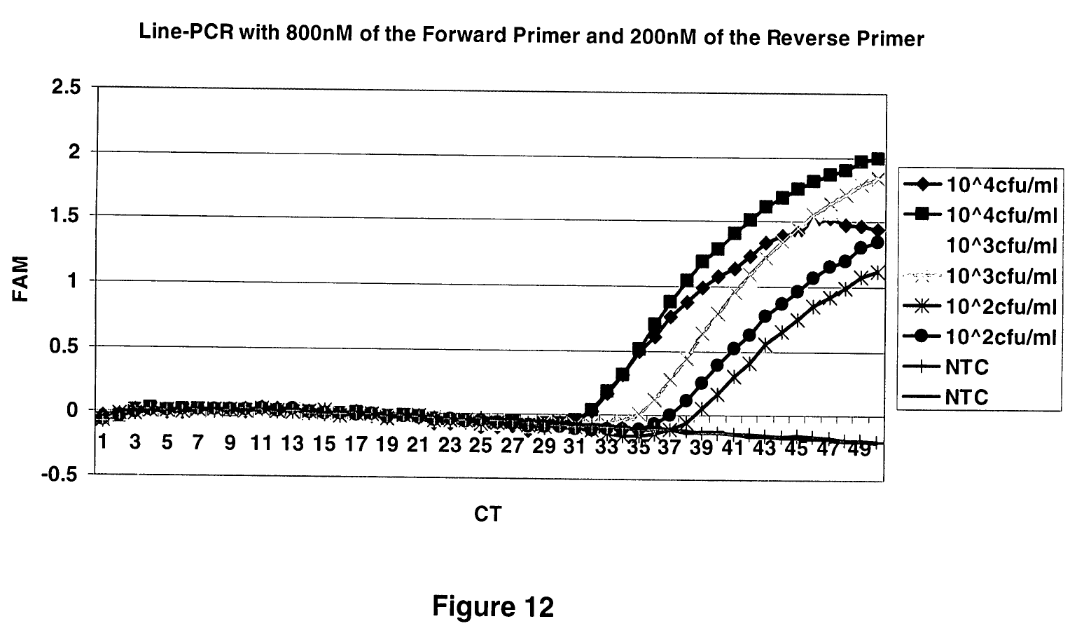

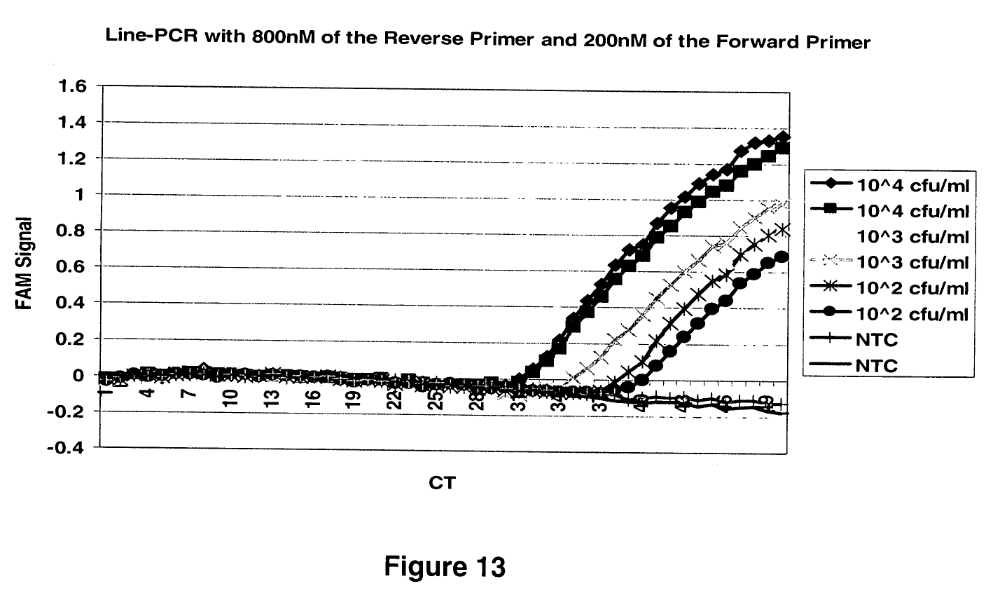

Four different Taqman™ PCR mastermixes were made to determine the optimal concentrations of primers to improve the amplification reaction. FIG. 10 shows the concentration of 200 nM of both primers and probes. FIG. 11 shows the results of a PCR reaction with 800 nM of primers and 200 nM of probes. FIG. 12 shows the results of a PCR mix with 800 nM of the forward Primer and 200 nM of the reverse primer and probe. Lastly, FIG. 13 displays the results of the PCR mix with 800 nM of the reverse primer and 200 nM of the forward primer and probe. Adding high concentrations of either forward or reverse primers increased the accuracy of amplification and detection of the specific genomic DNA bound to the solid matrix.

It will be apparent to the skilled artisan that the above description is merely exemplary and that the invention encompasses various modifications known to the skilled artisan.

We claim:

1. A method for the detection of a microorganism comprising:
(a) lysing a microorganism to release a nucleic acid molecule;
(b) contacting the nucleic acid molecule with a vessel coated with at least one metal oxide;
(c) allowing the nucleic acid molecule to attach to the metal oxide;
(d) amplifying, in the vessel, a fragment of the nucleic acid molecule attached to the metal oxide to generate an amplified product; and
(e) detecting, in the vessel, the amplified product.

2. The method of claim 1, wherein the microorganism is lysed in step (a) with a reagent that cleaves the peptidoglycan bonds on the outer membrane, a protease, a detergent, and an alkaline solution.

3. The method of claim 2, wherein the reagent that cleaves peptidoglycan bonds is lysozyme, lysostaphin, or mutanolysin.

4. The method of claim 3, wherein the reagent that cleaves peptidoglycan bonds is lysozyme.

5. The method of claim 2, wherein the protease is proteinase K, pronase E, achromopeptidase, proteinase R, proteinase T, subtilisin DY, an alkaline serine protease from Streptomyces griseus or Bacillus lichenformis, dispase, subtilisin Calsberg, subtilopeptidase A, or thermolysin.

6. The method of claim 5, wherein the protease is proteinase K.

7. The method of claim 2, wherein the detergent is t-octylphenoxypolyethoxyethanol (Triton X-100), polyoxyethylenesorbitan monolaurate (Tween-20), polyoxyethylenesorbitan monopalmitate (Tween-40), polyoxyethylenesorbitan monostearate (Tween-60), polyoxyethylenesorbitan monooleate (Tween-80), polyoxyethylenesorbitan monotrioleate (Tween-85), (octylphenoxy)polyethoxyethanol (IGEPAL CA-630), triethyleneglycol monolauryl ether (Brij 30), or sorbitan monolaurate (Span 20).

8. The method of claim 7, wherein the nonionic detergent is polyoxyethylenesorbitan monolaurate (Tween-20).

9. The method of claim 2, wherein the alkaline solution is 50 mM-500 mM KOH or NaOH.

10. The method of claim 1, wherein the microorganism is lysed in step (a) with a chaotropic salt or a cationic surfactant.

11. The method of claim 10, wherein the chaotropic agent is a guanidinium salt.

12. The method of claim 11, wherein the guanidinium salt is guanidine hydrochloride, guanidine thiocyanate, guanidinium chloride, guanidinium hydrochloride, or guanidinium isothiocyanate.

13. The method of claim 10, wherein the cationic surfactant is cetyltrimethylammonium bromide (CTAB), cetyltrimethylammonium chloride (CTACl), tetradecetyltrimethylammonium bromide (TTAB), tetradecetyltrimethylammonium chloride (TTACl), dodecyltrimethylammonium bromide (DTAB), dodecyltrimethylammonium chloride (DTACl), dodecylethyldimethylammonium chloride (DEDTAB), decyltrimethylammonium bromide (D10TAB), or dodecyltripheylphosphonium bromide (DTPB).

14. The method of claim 1, wherein the metal oxide is an oxide of aluminum, titanium, zirconium, hafnium, scandium, yttrium, lanthanum, vanadium, tantalum, chromium, molybdenum, tungsten, boron, gallium, indium, germanium, tin, zinc, nickel, magnesium, or iron.

15. The method of claim 14, wherein the metal oxide is aluminum oxide.

16. The method of claim 1, wherein the vessel is a tube, column, plate, or well.

17. The method of claim 16, wherein the vessel is a PCR tube.

18. The method of claim 1, where the attached nucleic acid molecule is washed with a buffer that comprises t-octylphenoxypolyethoxyethanol (Triton X-100), polyoxyethylenesorbitan monolaurate (Tween-20), polyoxyethylenesorbitan monopalmitate (Tween-40), polyoxyethylenesorbitan monostearate (Tween-60), polyoxyethylenesorbitan monooleate (Tween-80), polyoxyethylenesorbitan monotrioleate (Tween-85), (octylphenoxy)polyethoxyethanol (IGEPAL CA-630), triethyleneglycol monolauryl ether (Brij 30), sorbitan monolaurate (Span 20), methanol, ethanol, or isopropanol.

19. The method of claim 18, wherein the buffer comprises at least 5% w/w ethanol and at least 0.5% w/w polyoxyethylenesorbitan monolaurate (Tween-20).

20. The method of claim 1, wherein the amplified product is detected by hybridization to a probe.

21. The method of claim 20, wherein the amplified product is detected by hybridization to a labeled DNA, PNA, RNA, o-methyl RNA, LNA, or modified nucleotide probe.

22. The method of claim 21, wherein the probe is fluorescently labeled.

23. The method of claim 1, wherein the amplified product is detected with an intercalating dye.

24. The method of claim 1, wherein the nucleic acid molecule is amplified by the polymerase chain reaction (PCR).

25. The method of claim 24, wherein amplification is detected by hybridization using a probe.

26. The method of claim 25, wherein the amplified product is detected by hybridization to a labeled DNA, PNA, RNA, o-methyl RNA, LNA, or modified nucleotide probe.

27. The method of claim 26, wherein the probe is fluorescently labeled.

28. The method of claim 24, wherein amplification is detected with an intercalating dye.

29. The method of claim 24, wherein the amplified product is detected during amplification.

30. The method of claim 24, wherein the nucleic acid molecule is amplified with a fluorescently labeled primer.

31. A kit for detecting nucleic acids in biological sample comprising:
(a) a vessel coated with at least one metal oxide;
(b) instructions for binding a nucleic acid molecule to the metal oxide; and
(c) instructions for amplifying and detecting, in the vessel, a nucleic acid molecule bound to the metal oxide.

32. The kit of claim 31, further comprising primers for amplifying a nucleic acid molecule bound to the metal oxide.

33. The kit of claim 31, further comprising a fluorescent probe.

34. The kit of claim 31, further comprising a lysozyme, a protease, a nonionic detergent, and an alkaline solution.

35. The kit of claim 31, wherein the metal oxide is an oxide of aluminum, titanium, zirconium, hafnium, scandium, yttrium, lanthanum, vanadium, tantalum, chromium, molybdenum, tungsten, boron, gallium, indium, germanium, tin, zinc, nickel, magnesium, or iron.

36. The kit of claim 35, wherein the metal oxide is aluminum oxide.

37. The kit of claim 31, wherein the vessel is a tube, column, plate, or well.

38. The kit of claim 36, wherein the vessel is a PCR tube.

39. A kit for detecting nucleic acids in biological sample comprising:
(a) a vessel coated with at least one metal oxide and
(b) instructions for practicing the method of claim 1.

40. The kit of claim 39, further comprising primers for amplifying a nucleic acid molecule bound to the metal oxide.

41. The kit of claim 39, further comprising a fluorescent probe.

42. The kit of claim 39, further comprising a lysozyme, a protease, a nonionic detergent, and an alkaline solution.

43. The kit of claim 39, wherein the metal oxide is an oxide of aluminum, titanium, zirconium, hafnium, scandium, yttrium, lanthanum, vanadium, tantalum, chromium, molybdenum, tungsten, boron, gallium, indium, germanium, tin, zinc, nickel, magnesium, or iron.

44. The kit of claim 43, wherein the metal oxide is aluminum oxide.

45. The kit of claim 39, wherein the vessel is a tube, column, plate, or well.

46. The kit of claim 45, wherein the vessel is a PCR tube.

47. A method for the detection of a nucleic acid comprising:
(a) attaching a nucleic acid molecule to a metal oxide coated onto a vessel;
(b) amplifying, in the vessel, a fragment of the nucleic acid molecule to generate an amplified product; and
(c) detecting, in the vessel, the amplified product.

48. The method of claim 47, wherein the metal oxide is an oxide of aluminum, titanium, zirconium, hafnium, scandium, yttrium, lanthanum, vanadium, tantalum, chromium, molybdenum, tungsten, boron, gallium, indium, germanium, tin, zinc, nickel, magnesium, or iron.

49. The method of claim 47, wherein the metal oxide is aluminum oxide.

50. The method of claim 49, wherein the vessel is a tube, column, plate, or well.

51. The method of claim 50, wherein the vessel is a PCR tube.

52. The method of claim 47, wherein the amplified product is detected by hybridization to a probe.

53. The method of claim 52, wherein the amplified product is detected by hybridization to a labeled DNA, PNA, RNA, o-methyl RNA, LNA, or modified nucleotide probe.

54. The method of claim 53, wherein the probe is fluorescently labeled.

55. The method of claim 47, wherein the amplified product is detected with an intercalating dye.

56. The method of claim 47, wherein the nucleic acid molecule is amplified by the polymerase chain reaction (PCR).

57. The method of claim 56, wherein amplification is detected by hybridization using a probe.

58. The method of claim 57, wherein the amplified product is detected by hybridization to a labeled DNA, PNA, RNA, o-methyl RNA, LNA, or modified nucleotide probe.

59. The method of claim 58, wherein the probe is fluorescently labeled.

60. The method of claim 56, wherein amplification is detected with an intercalating dye.

61. The method of claim 56, wherein the amplified product is detected during amplification.

62. The method of claim 56, wherein the nucleic acid molecule is amplified with a fluorescently labeled primer.


SEQUENCE LISTING (4 sequences)

SEQ ID NO: 1 - DNA, 16 bp, Listeria monocytogenes
tcgtgcgcttctaggt

SEQ ID NO: 2 - DNA, 18 bp, Listeria monocytogenes
tgctttagttgcgatgga

SEQ ID NO: 3 - DNA, 28 bp, Listeria monocytogenes
tatgagtcgccttagctacaatgtatct

SEQ ID NO: 4 - DNA, 464 bp, Listeria monocytogenes
aattactagatcaaactgctacaggtgctgctactcaagtaagcatccaagcgtctgataaagctaatgacttaatcaatatcgatcttttcaacgctaaaggtctttctgctggaacaatcactttaggtagtggttctacagttgctggttatagtgcattatccgttgctgatgctgattcttctcaacaagcaactgaagctattgatgaattaatcaataacatttctaacggtcgtgcgcttctaggtgctggtatgagtcgccttagctacaatgtatctaacgtgaacaatcaatccatcgcaactaaagcatctgcttcatccattgaagatgcagatatggctgctgaaatgtccgaaatgactaaatacaaaattcttacacaaacttctatcagcatgctttctcaagcaaaccaaacaccgcaaatgttaactcaattaattaacagctaa